(19) United States
(12) Patent Application Publication
(10) Pub. No.: US 2026/0253680 A1
(43) Pub. Date: Aug. 27, 2026

(54) DEEP LEARNING-BASED MOLECULAR DESIGN SYSTEM, AND DEEP LEARNING-BASED MOLECULAR DESIGN METHOD

(71) Applicant: KOREA UNIVERSITY RESEARCH AND BUSINESS FOUNDATION, Seoul (KR)

(72) Inventors: Sungnam PARK, Seoul (KR); Joonyoung JOUNG, Seoul (KR); Minhi HAN, Seoul (KR); Minseok JEONG, Hanam-si (KR); Dong Hoon CHOI, Seoul (KR)

(73) Assignee: KOREA UNIVERSITY RESEARCH AND BUSINESS FOUNDATION, Seoul (KR)

(21) Appl. No.: 18/875,905

(22) PCT Filed: Jun. 22, 2023
(86) PCT No.: PCT/KR2023/008705

§ 371 Date: Dec. 17, 2024

(30) Foreign Application Priority Data
Jun. 23, 2022 (KR) 10-2022-0076504

(51) Int. Cl. G16C 20/50; G16C 20/70

(52) U.S. Cl. CPC G16C 20/50; G16C 20/70

(57) ABSTRACT

A deep learning-based molecular design system according to the present invention comprises: a vectorizer which receives and vectorizes molecular information of an i-th molecule, surrounding molecular information, and molecular property information; an feature extractor which extracts molecular features of the i-th molecule from the vectorized molecular information of the i-th molecule, extracts surrounding molecular feature from the vectorized surrounding molecular information, and extracts molecular property feature from the vectorized molecular property information; an integrated feature extractor which extracts integrated feature of the i-th molecule using an integrated feature extraction algorithm, which is a neural network algorithm that receives the molecular feature of the i-th molecule, surrounding molecular feature, and molecular property feature as inputs; a molecular design probability calculator which extracts a molecular design probability vector for molecular design on the basis of the i-th molecule using a molecular design probability calculation algorithm, which is a neural network algorithm that receives the integrated feature of the i-th molecule as inputs; and a molecular designer which extracts molecular information about the (i+1)-th molecule on the basis of the molecular design probability vector or outputs a design stop command to output a final molecule, wherein i is an integer greater than or equal to 1.

### TECHNICAL FIELD

The present invention relates to a deep learning-based molecular design system and a deep learning-based molecular design method. Specifically, the present invention relates to a deep learning-based molecular design system and a deep learning-based molecular design method for designing a molecule having specified molecular properties as well as considering the effect of surrounding molecules.

### BACKGROUND ART

Many material molecules are being developed to develop materials suitable for a purpose. In general, researchers want to develop molecules that are predicted to have desired molecular properties based on their experience and theories, but it is difficult to develop molecules having desired molecular properties due to the limitations of researchers' experience and theories.

Therefore, molecules having desired molecular properties are developed through various trials and errors, but various problems arise, such as the large amount of time and cost required.

Meanwhile, there have been various attempts recently to design molecules having desired molecular properties using machine learning or deep learning methods, but the accuracy of molecular design is low because the surrounding environment of the molecule is not considered.

Accordingly, there is a need for a technology capable of not only reducing time and cost but also accurately designing molecules with desired properties while considering the surrounding environment.

The present invention is derived from research conducted as a part of by the Ministry of Education's Science and Engineering Research Institute Support Project (Project Number: 1345347024, Project Number: 2019R1A6A1A11044070, Project Name: π-Electronic-based Energy Environment Innovative Materials Research, Project Management Organization: Korea University Industry-University Cooperation foundation, Project Execution Organization: Korea University Industry-Academic Cooperation Center, Research Period: 2022 Mar. 1-2023 Feb. 28. Contribution rate: 50%) and personal basic research (Ministry of Science and ICT) (Project Number: 1711153079, Project number: 2022R1A2C1003627, Project name: Deep learning-based prediction of molecular properties and generation of new molecular structures, project management organization: Korea Research Foundation, Project Execution Organization; Korea University Industry-University Cooperation foundation, Project period; 2022 Mar. 1-2023 Feb. 28, Contribution rate: 50%). The Korean government has no property interest in any aspect of this invention.

### DETAILED DESCRIPTION OF THE INVENTION

### Techinical Problem

An object of the present invention is to provide a deep learning-based molecular design system and a deep learning-based molecular design method for designing a molecule having desired molecular properties by considering a surrounding environment (or, surrounding molecules).

### Technical Solution

According to an embodiment to the present invention, a deep learning-based molecular design system includes a vectorizer configured to receive and vectorize i-th molecular information, surrounding molecular information, and molecular property information, a feature extractor configured to extract an i-th molecular feature from the vectorized the i-th molecular information, extract a surrounding molecular feature from the vectorized surrounding molecular information, and extract a molecular property feature from the vectorized molecular property information, an integrated feature extractor configured to extract an integrated feature of the i-th molecule using an integrated feature extraction algorithm which is a neural network algorithm that receives the i-th molecular feature, the surrounding molecular feature, and the molecular property feature as an input, a molecular design probability calculator configured to extract a molecular design probability vector for molecular design based on the i-th molecule using a molecular design probability calculation algorithm, which is a neural network algorithm that receives the integrated feature of i-th molecule as an input, and a molecular designer configured to extract (i+1)-th molecular information based on the molecular design probability vector or outputting a design stop command to output a final molecule, i being an integer greater than or equal to 1.

According to an embodiment to the present invention, the vectorizer may include a molecular information vectorizer configured to receive the i-th molecular information in form of SMILES (Simplified molecular-Input Line-Entry System) representation, and vectorize the i-th molecular information using at least one of a molecular fingerprint, a molecular descriptor, an image of a chemical structural formula, a molecular graph, molecular coordinates, and a SMILES code, a surrounding molecular information vectorizer configured to receive the surrounding molecular information in the form of the SMILES (Simplified molecular-Input Line-Entry System) representation, and vectorize the surrounding molecular information using at least one of the molecular fingerprint, the molecular descriptor, the image of a chemical structural formula, the molecular graph, the molecular coordinates, and the SMILES code, and a molecular property information vectorizer configured to receive the molecular property information in form of a string or a set of real values and vectorize the molecular property information using at least one of tokenization, normalization, and one-hot encoding.

According to an embodiment to the present invention, the feature extractor may include a molecular feature extractor configured to extract the molecular feature of the i-th molecule using a molecular feature extraction algorithm, which is a neural network algorithm that receives the vectorized i-th molecular information as an input, a surrounding molecular feature extractor configured to extract a surrounding molecular feature using a surrounding molecular feature extraction algorithm, which is a neural network algorithm that receives the vectorized surrounding molecular information as an input, and a molecular property feature extractor configured to extract the molecular property feature using a molecular property feature extraction algorithm, which is a neural network algorithm that receives the vectorized molecular property information as an input.

According to an embodiment to the present invention, the molecular information may include information about a chemical structural formula, the surrounding molecular information may include information about one or more solvents, and the molecular property information may include information about at least one of structural, chemical, physical, spectroscopic, electrochemical, and reactivity of the molecule.

According to an embodiment to the present invention, molecular information of an initial molecule (where i=1) may include information about the chemical structural formula provided by a user or provided by the deep learning-based molecular design system.

According to an embodiment to the present invention, the molecular designer may extract the (i+1)-th molecular information for designing the (i+1)-th molecule according to a probability value calculated using one of elements constituting the molecular design probability vector; and the (i+1)-th molecular information may include information about the chemical structural formula of the (i+1)-th molecule, which is designed by either binding a single atom to one of atoms constituting the i-th molecule, or by adding a bond that connects the atoms constituting the i-th molecule.

According to an embodiment to the present invention, the molecular designer may output the design stop command according to a probability value calculated using one of the elements constituting the molecular design probability vector to determine the i-th molecule as the final molecule.

According to an embodiment to the present invention, the molecular feature extraction algorithm, the surrounding molecular feature extraction algorithm, the molecular property feature extraction algorithm, the integrated feature extraction algorithm, and the molecular design probability calculation algorithm include at least one hidden layer.

According to an embodiment to the present invention, a deep learning-based molecular design method includes receiving and vectorizing, by a vectorizer, i-th molecular information, surrounding molecular information, and molecular property information, extracting, by an feature extractor, a molecular feature from the vectorized i-th molecular information, extracting a surrounding molecular feature from the vectorized surrounding molecular information, and extracting a molecular property feature from the vectorized molecular property information, extracting, by an integrated feature extractor, an integrated feature of the i-th molecule using an integrated feature extraction algorithm which is a neural network algorithm that receives the i-th molecular feature, the surrounding molecular feature, and the molecular property feature as an input, extracting, by a molecular design probability calculator, a molecular design probability vector for molecular design based on the i-th molecule using a molecular design probability calculation algorithm, which is a neural network algorithm that receives the integrated feature of the i-th molecule as an input, and extracting, by a molecular designer, (i+1)-th molecular information based on the molecular design probability vector or output a design stop command to output a final molecule, i being an integer greater than or equal to 1,

According to an embodiment to the present invention, there is provided a non-transitory computer-readable recording medium having recorded thereon a program for executing the deep learning-based molecular design method.

### Advantageous Effects of the Invention

The deep learning-based molecular design system and deep learning-based molecular design method according to the present invention may design molecules having desired molecular properties in consideration of the surrounding molecules to improve the accuracy of molecular design.

Further, the deep learning-based molecular design system and deep learning-based molecular design method according to the present invention may design molecules with desired molecular properties based on user-provided information, thereby reducing trial and error in the molecular design process, as well as reducing time and development costs.

### DESCRIPTION OF THE DRAWINGS

FIG. 1 is a diagram illustrating a configuration of a deep learning-based molecular design system according to an embodiment of the present invention.

FIG. 2A is a diagram of an implementation example of an feature extractor according to an embodiment of the present invention. FIG. 2B is a diagram of an implementation example of an integrated feature extractor according to an embodiment of the present invention. FIG. 2C is a diagram of an implementation example of a molecular design probability calculator, according to an embodiment of the present invention. FIG. 2D is a diagram of an implementation example of a molecular designer according to an embodiment of the present invention.

FIG. 3A is a diagram illustrating an implementation example of designing a final molecule in a deep learning-based molecular design system according to an embodiment of the present invention. FIG. 3B is a diagram illustrating an implementation example of designing a final molecule in a deep learning-based molecular design system according to another embodiment of the present invention.

FIG. 4 is a diagram illustrating a process for designing a final molecule according to a molecular design probability vector using benzene as an initial molecule according to an embodiment of the present invention.

FIG. 5A is a diagram illustrating the result of designing a final molecule based on molecular information, surrounding molecular information, and molecular property information according to an embodiment of the present invention. FIG. 5B is a diagram illustrating the result of designing a final molecule based on molecular information, surrounding molecular information, and molecular property information according to another embodiment of the present invention. FIG. 5C is a diagram illustrating the result of designing a final molecule based on molecular information, surrounding molecular information, and molecular property information according to another embodiment of the present invention. FIG. 5D is a diagram illustrating the result of designing a final molecule based on molecular information, surrounding molecular information, and molecular property information according to another embodiment of the present invention.

FIG. 6 is a flowchart of a deep learning-based molecular design method according to an embodiment of the present invention.

### BEST MODE

Hereinafter, with reference to the accompanying drawings, various embodiments of the present invention will be described in detail such that those of ordinary skill in the art can easily carry out the present invention. The present invention may be embodied in several different forms and is not limited to the embodiments described herein.

In order to clearly explain the present invention, parts irrelevant to the present invention are omitted, and the same reference numerals are assigned to the same or similar elements throughout the specification. Accordingly, the reference numerals described above may be used in other drawings as well.

Further, in the drawings, a size, and a thickness of each element are arbitrarily illustrated for convenience of description, and the present invention is not necessarily limited to those illustrated in the drawings. In the drawings, the thicknesses of layers and regions are enlarged for clarity.

The expression “the same” means to “substantially the same”. That is, it may be the same degree to the extent that ordinary knowledge those of ordinary skill in the art can convince that they are the same. Other expressions may be expressions in which” is omitted.

In addition, throughout the specification, unless explicitly described to the contrary, the word “comprise” and variations such as “comprises” or “comprising” will be understood to imply the inclusion of stated elements but not the exclusion of any other elements. As used throughout this specification, ‘~unit’ is a unit that processes at least one function or operation, and may refer to, for example, a software component, FPGA or or a hardware component. A function provided by ‘~unit’ may be performed separately by a plurality of components, or may be integrated with other additional components. The term ‘~unit’ in this specification is not necessarily limited to software or hardware, and may be configured to reside in an addressable storage medium, or may also be configured to reproduce one or more processors. Hereinafter, embodiments of the present invention will be described in detail with reference to the accompanying drawings.

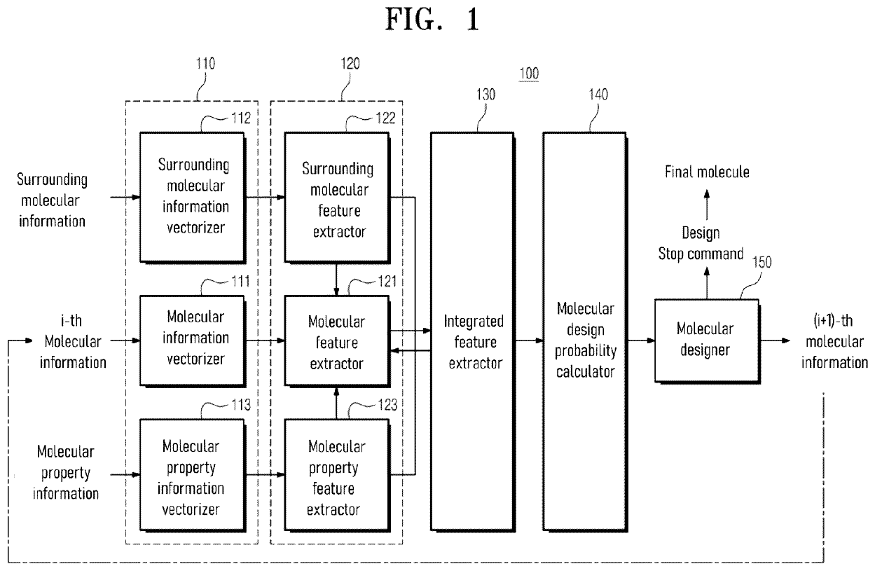

FIG. 1 is a diagram illustrating the configuration of a deep learning-based molecular design system according to an embodiment of the present invention.

The deep learning-based molecular design system 100 according to an embodiment of the present invention may include a vectorizer 110, an feature extractor 120, an integrated feature extractor 130, a molecular design probability calculator 140, and a molecular designer 150.

The vectorizer 110 may include a molecular information vectorizer 111, a surrounding molecular information vectorizer 112, and a molecular property information vectorizer 113. The feature extractor 120 may include a molecular feature extractor 121, a surrounding molecular feature extractor 122, and a molecular property feature extractor 123.

The vectorizer 110 may receive and vectorize i-th molecular information (where i is an integer greater than or equal to 1), surrounding molecular information, and molecular property information.

Specifically, the molecular information vectorizer 111 may receive the i-th molecular information in the form of a SMILES (Simplified Molecular-Input Line-Entry System) representation and vectorize the molecular information using at least one of a molecular fingerprint, a molecular descriptor, an image of a chemical structural formula, a molecular graph, molecular coordinates, and a SMILES code.

In this case, SMILES (Simplified Molecular-Input Line-Entry System) refers to a method of representing chemical structure information such as chemical components, bond types, aromaticity, and the presence or absence of branches as a string of ASCII codes.

The surrounding molecular information vectorizer 112 may receive the surrounding molecular information in the form of the SMILES (Simplified molecular-Input Line-Entry System) representation, like the molecular information vectorizer 111 described above, and vectorize the surrounding molecular information using at least one of the following representation methods: a molecular fingerprint, a molecular descriptor, an image of a chemical structural formula, a molecular graph, molecular coordinates, and a SMILES code.

The molecular property information vectorizer 113 may receive the molecular property information in the form of a string or a set of real values and vectorize the molecular property information using at least one of tokenization, normalization, and one-hot encoding.

I-th molecular information may include information about the chemical structural formula of i-th molecule. For example, molecular information of an initial molecule (where i=1) may include information about the chemical structural formula of a specified molecule which is provided by a user or include information about the chemical structural formula provided by the deep learning-based molecular design system.

Further, the surrounding molecular information may include information about one or more solvents that are surrounding environment in which a molecule is designed (hereinafter referred to as the surrounding molecules).

Specifically, when the surrounding molecules have a gas phase, the surrounding molecules be absent or may include information about a gas molecule. When the surrounding molecules have a liquid phase, the surrounding molecules include information about a single solvent or a plurality of solvents, such as a cosolvent. When the surrounding molecules have a solid phase, the surrounding molecules may include information about a single solvent or a plurality of solvents, such as a cosolvent, a matrix, and a host.

The molecular property information may also include information about at least one of structural, chemical, physical, spectroscopic, electrochemical, or reactivity of a molecule.

For example, the molecular property information may include only information about one of the structural, chemical, physical, spectroscopic, electrochemical, and reactivity of the molecule. Alternatively, the molecular property information may include information about at least two of the structural, chemical, physical, spectroscopic, electrochemical, and reactivity of the molecule.

The molecular feature extractor 121 may extract an i-th molecular feature from the vectorized i-th molecular information.

The molecular feature extractor 121 may pre-store a molecular feature extraction algorithm in the form of a neural network algorithm. The molecular feature extractor 121 may extract the i-th molecular feature by inputting the vectorized molecular information of the i-th molecule into the molecular feature extraction algorithm in the form of a neural network algorithm.

The surrounding molecular feature extractor 122 may extract surrounding molecular information from the vectorized surrounding molecular information.

The surrounding molecular feature extractor 122 may store a surrounding molecular feature extraction algorithm in the form of a neural network algorithm in advance. The surrounding molecular feature extractor 122 may extract the surrounding molecular feature by inputting the vectorized surrounding molecular information into the surrounding molecular feature extraction algorithm in the form of a neural network algorithm.

The molecular property feature extractor 123 may extract a molecular property feature from the vectorized molecular property information.

The molecular property feature extractor 123 may store a molecular property feature extraction algorithm in the form of a neural network algorithm in advance. The molecular property feature extractor 123 may extract the molecular property feature by inputting the vectorized molecular property information into the molecular property feature extraction algorithm in the form of a neural network algorithm.

Further, the molecular feature extractor 121 may extract the i-th molecular feature, by receiving the surrounding molecular feature extracted from the surrounding molecular feature extractor 122, the molecular property feature extracted from the molecular property feature extractor 123, and an integrated feature of the i-th molecule extracted from the integrated feature extractor 130 according to the molecular feature extraction algorithm used, the integrated feature extractor 130 being to be described below.

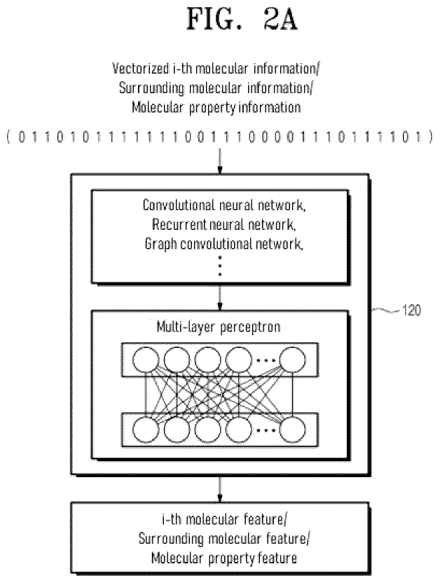

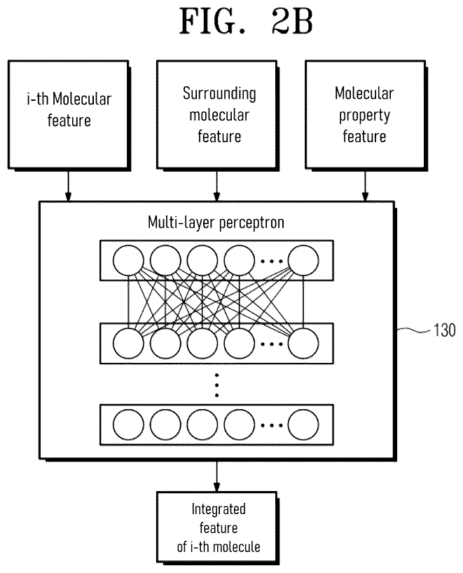

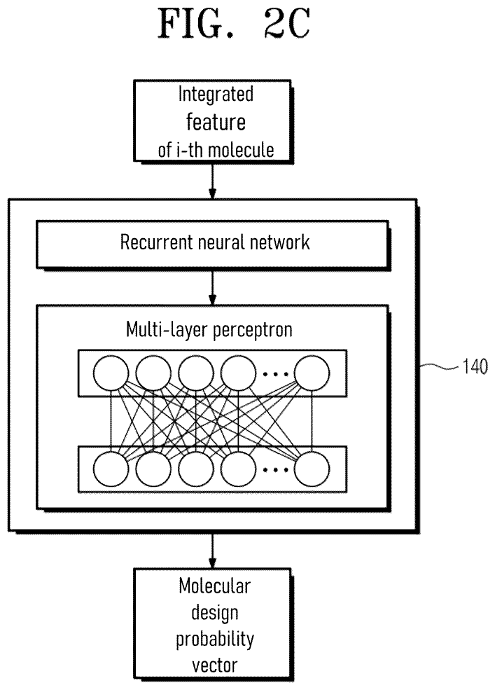

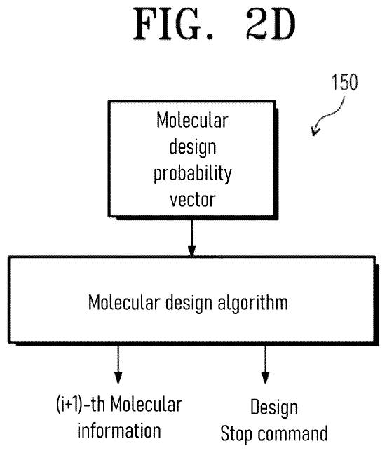

A process of extracting the i-th molecular feature from the molecular feature extractor 121, a process of extracting the surrounding molecular feature from the surrounding molecular feature extractor 122, and a process of extracting the molecular property feature from the molecular property feature extractor 123 will be described in detail with reference to FIG. 2A below.

The integrated feature extractor 130 may extract the integrated feature of the i-th molecule using the i-th molecular feature, the surrounding molecular feature, and the molecular property feature.

Specifically, the integrated feature extractor 130 may store an integrated feature extraction algorithm in the form of a neural network algorithm in advance. The integrated feature extractor 130 may extract the integrated feature of the i-th molecule by inputting the i-th molecular feature, the surrounding molecular feature, and the molecular property feature, which are provided by the feature extractor 120, into the integrated feature extraction algorithm in the form of a neural network algorithm.

The process of extracting the integrated feature of the i-th molecule in the integrated feature extractor 130 will be described in detail with reference to FIG. 2B below.

The molecular design probability calculator 140 may output a molecular design probability vector for molecular design based on the i-th molecule using the integrated feature of the i-th molecule.

Specifically, the molecular design probability calculator 140 may store a molecular design probability calculation algorithm in the form of a neural network algorithm in advance. The molecular design probability calculator 140 may extract a molecular design probability vector for molecular design based on the i-th molecule by inputting the integrated feature of the i-th molecule provided by the integrated feature extractor 130 into the molecular design probability calculation algorithm in the form of a neural network algorithm.

A process of extracting the molecular design probability vector for molecular design based on the i-th molecule in the molecular design probability calculator 140 will be described in detail with reference to FIG. 2C below.

The molecular designer 150 may extract (i+1)-th molecular information for designing the (i+1)-th molecule according to probability values calculated using the elements constituting the molecular design probability vector extracted by the molecular design probability calculator 140.

In this case, the (i+1)-th molecular information includes information about the chemical structural formula of the (i+1)-th molecule, which is designed by either binding a single atom to one of the atoms constituting the i-th molecule, or by adding a bond that connects the atoms constituting the i-th molecule.

Alternatively, the molecular designer 150 may determine and output the i-th molecule as the final molecule by outputting a design stop command based on the probability values calculated using the elements constituting the molecular design probability vector extracted by the molecular design probability calculator 140.

A process of extracting (i+1)-th molecular information using the molecular design probability vector or outputting the design stop command to determine the final molecule in the molecular designer 150 will be described in detail with reference to FIG. 2D below.

When (i+1)-th molecular information is extracted based on the molecular design probability vector in the molecular designer 150 described above, (i+1)-th molecular information may be input to the molecular information vectorizer 111, and the process described above may be repeatedly performed until the design stop command is output from the molecular designer 150 to design the molecule and determine the final molecule.

As described above in FIG. 1, the deep learning-based molecular design system 100 according to an embodiment of the present invention may significantly reduce development time and cost by designing a final molecule with specific molecular properties while considering surrounding molecules.

FIG. 2A is a diagram of an implementation example of a feature extractor according to an embodiment of the present invention. FIG. 2B is a diagram of an implementation example of an integrated feature extractor according to an embodiment of the present invention. FIG. 2C is a diagram of an implementation example of a molecular design probability calculator, according to an embodiment of the present invention. FIG. 2D is a diagram of an implementation example of a molecular designer according to an embodiment of the present invention.

Referring to FIGS. 2A to 2D, the molecular feature extraction algorithm, the surrounding molecular feature extraction algorithm, the molecular property feature extraction algorithm, the integrated feature extraction algorithm, and the molecular design probability calculation algorithm implemented in the feature extractor 120, the integrated feature extractor 130, the molecular design probability calculator 140, and the molecular designer 150 according to an embodiment of the present invention may be a neural network algorithm including at least one hidden layer.

According to an embodiment of the present invention, a process of extracting i-th molecular information in the molecular feature extractor 121, a process of extracting surrounding molecular feature in the surrounding molecular feature extractor 122, and a process of extracting molecular property feature in the molecular property feature extractor 123 may be performed independently of each other.

Hereinafter, the surrounding molecular feature extractor 122 of the present invention will be described as an example below with reference to FIG. 2A.

Referring to FIG. 2, the surrounding molecular feature extraction algorithm pre-stored in the surrounding molecular feature extractor 122 may be in the form of a neural network algorithm including one or more hidden layers and may be implemented with a multi-layer perceptron (MLP).

In this case, as the surrounding molecular feature extraction algorithm, an additional algorithm may be applied according to the vectorization format of the surrounding molecular information input to the surrounding molecular feature extraction algorithm of the surrounding molecular feature extractor 122, in addition to the Multi-Layer Perceptron (MLP) as described above.

For example, when the vectorization format of the surrounding molecular information input to the surrounding molecular feature extraction algorithm of the surrounding molecular feature extractor 122 is an image format, the additional algorithm may be a convolutional neural network (CNN). Alternatively, when the vectorization format is a string format, the additional algorithm may be a Recurrent Neural Network (RNN). Alternatively, when the vectorization format is a graph format, the additional algorithm may be a Graph Convolutional Network (GCN).

On the other hand, the Multi-Layer Perceptron (MLP) described above may be applied first before the additional algorithms described above are applied, or the Multi-Layer Perceptron (MLP) described above may be applied after the additional algorithms described above are applied.

Alternatively, the additional algorithms described above may be applied in combination before or after the Multi-Layer Perceptron (MLP) described above is applied.

In other words, the surrounding molecular feature extraction algorithm may be implemented with the Multi-Layer Perceptron (MLP), a combination of the Multi-Layer Perceptron (MLP) and an additional algorithm, or a combination of the Multi-Layer Perceptron (MLP) and an additional algorithm, or a combination of the combination of the Multi-Layer Perceptron (MLP) and an additional algorithm.

The surrounding molecular feature extractor 122 may extract the surrounding molecular feature by inputting the vectorized surrounding molecular information into the surrounding molecular feature extraction algorithm described above, which may be in the form of a neural network algorithm.

Since the process of extracting the i-th molecular feature in the molecular feature extractor 121 and the process of extracting the molecular property feature in the molecular property feature extractor 123 are substantially the same or similar to the process of extracting the surrounding molecular feature in the surrounding molecular feature extractor 122 described above, the redundant description will be omitted.

On the other hand, the process of extracting the i-th molecular feature in the molecular feature extractor 121 may extract the i-th molecular feature by additionally receiving the surrounding molecular feature extracted by the surrounding molecular feature extractor 122, the molecular property feature extracted by the molecular property feature extractor 123, and the integrated feature of the i-th molecule extracted by the integrated feature extractor 130, which will be described with reference to FIG. 2B below. Referring to FIG. 2B, the integrated feature extraction algorithm pre-stored in the integrated feature extractor 130 may be in the form of a neural network algorithm including one or more hidden layers, and may be implemented with at least one Multi-Layer Perceptron (MLP).

The integrated feature extractor 130 may extract the integrated feature of the i-th molecule by inputting the i-th molecular feature, the surrounding molecular feature, and the molecular property feature, which are provided by the feature extractor 120, into the integrated feature extraction algorithm described above, which is in the form of a neural network algorithm.

Referring to FIG. 2C, the molecular design probability calculation algorithm pre-stored in the molecular design probability calculator 140 may be in the form of a neural network algorithm including one or more hidden layers and may be implemented with a Multi-Layer Perceptron (MLP).

In this case, as the molecular design probability calculation algorithm of the molecular design probability calculator 140, an additional algorithm may be applied in addition to the Multi-Layer Perceptron (MLP) described above.

For example, the molecular design probability calculation algorithm of the molecular design probability calculator 140 may include an additional algorithm in the form of a recurrent neural network (RNN) in addition to the Multi-Layer Perceptron (MLP) described above.

On the other hand, the Multi-Layer Perceptron (MLP) described above may be applied first before the additional algorithms described above are applied, or the Multi-Layer Perceptron (MLP) described above may be applied after the additional algorithms described above are applied.

The molecular design probability calculator 140 may extract a molecular design probability vector for molecular design based on the i-th molecule by inputting the integrated feature of the i-th molecule provided by the integrated feature extractor 130 into the molecular design probability calculation algorithm described above in the form of a neural network algorithm.

In this case, at least one or more elements may constitute the molecular design probability vector. Each of the elements constituting the molecular design probability vector may represent a probability value for designing the (i+1)-th molecule by binding a single atom to one of atoms constituting the i-th molecule, a probability value for designing the (i+1)-th molecule by adding a bond between the atoms constituting the i-th molecule, and a probability value for determining the i-th molecule as the final molecule by outputting a design stop command.

Referring to FIG. 2D, the molecular designer 150 may extract molecular information of an (i+1)-th molecule for designing the (i+1)-th molecule according to probability values calculated using the elements constituting the molecular design probability vector extracted by the molecular design probability calculator 140.

Specifically, as described above in FIG. 2C, the molecular designer 150 may select one of elements constituting the molecular design probability vector extracted by the molecular design probability calculator 140 and calculate a probability value.

The molecular designer 150 may extract (i+1)-th molecular information for designing the (i+1)-th molecule by bonding a single atom to one of the atoms constituting the i-th molecule or adding a bond connecting the atoms constituting the i-th molecule according to the probability value described above.

The molecular designer 150 may determine and output the i-th molecule as the final molecule by outputting a design stop command according to the probability value described above.

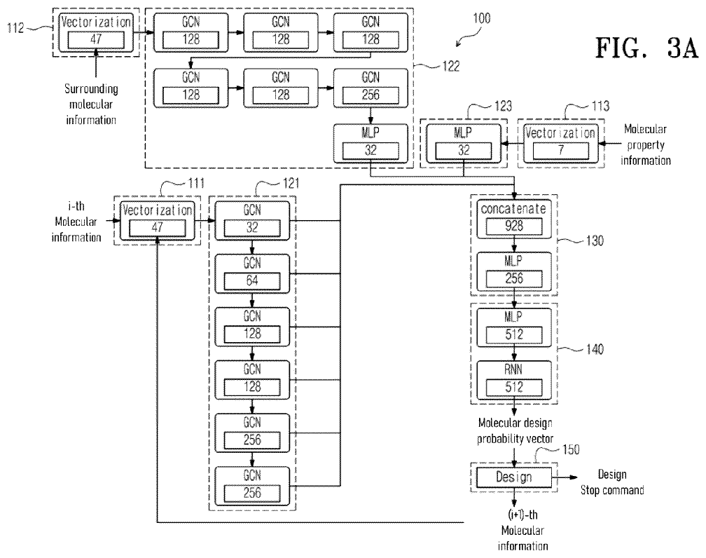

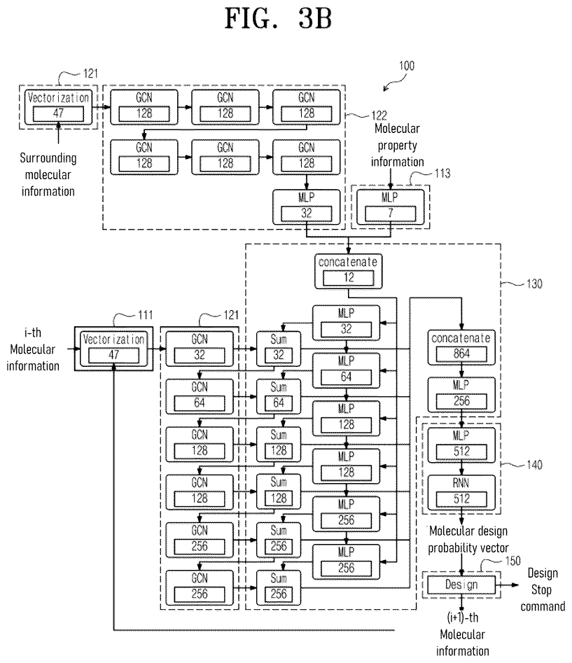

FIG. 3A is a diagram illustrating an implementation example of designing a final molecule in a deep learning-based molecular design system according to an embodiment of the present invention. FIG. 3B is a diagram illustrating an implementation example of designing a final molecule in a deep learning-based molecular design system according to another embodiment of the present invention.

First, with reference to FIG. 3A, an implementation example of designing a final molecule in a deep learning-based molecular design system according to an embodiment of the present invention will be described.

The molecular information vectorizer 111 may receive and vectorize i-th molecular information. The surrounding molecular information vectorizer 112 may receive and vectorize surrounding molecular information.

In this case, i-th molecular information vectorized by the molecular information vectorizer 111 and the surrounding molecular information vectorized by the surrounding molecular information vectorizer 112 may be vectorized using the representation method of a molecular graph.

The molecular property information vectorizer 113 may receive and vectorize the molecular property information.

The i-th molecular information vectorized by the molecular information vectorizer 111 may be input to the molecular feature extractor 121. In this case, i-th molecular information molecule may be sequentially passed through GCNs (Graph Convolutional Networks) of six layers including 32, 64, 128, 128, 256, and 256 nodes (or, elements) respectively, and a total of six features of the i-th molecule may be extracted as the output value of the GCN (Graph Convolutional Network).

The surrounding molecular information vectorized by the surrounding molecular information vectorizer 112 may be input to the surrounding molecular feature extactor 122. In this case, the surrounding molecular information may be sequentially passed through a Graph Convolutional Network (GCN) consisting of 128, 128, 128, 128, 128, 128, 128, and 256 nodes (or elements) and a Multi-Layer Perceptron (MLP) consisting of 32 nodes (or elements) to extract the surrounding molecular feature.

The molecular property information vectorized by the molecular property information vectorizer 113 may be input to the molecular property feature extractor 123. In this case, the molecular property information may be passed through a multi-layer perceptron (MLP) including 32 nodes (or, elements) to extract the molecular property feature.

The i-th molecular feature extracted by the molecular feature extractor 121, the surrounding molecular feature extracted by the surrounding molecular feature extractor 122, and the molecular property feature extracted by the molecular property feature extractor 123 may be input to the integrated feature extractor 130 and concatenated with each other.

The i-th molecular feature, surrounding molecular feature, and molecular property feature input to the integrated feature extractor 130 may be passed through a multi-layer perceptron (MLP) consisting of 256 nodes (or, elements) to extract the integrated feature of the i-th molecule.

The integrated feature of the i-th molecule extracted by the integrated feature extractor 130 may be input to the molecular design probability calculator 140.

The integrated feature of the i-th molecule input to the molecular design probability calculator 140 may be passed through a multi-layer perceptron (MLP) consisting of 512 nodes (or, elements) and a recurrent neural network (RNN) consisting of 512 nodes (or, elements) to extract a molecular design probability vector for molecular design based on the i-th molecule.

The molecular design probability vector extracted by the molecular design probability calculator 140 may be input to the molecular designer 150.

The molecular designer 150 may calculate probability values by using the elements constituting the input molecular design probability vector as weights respectively and select one of the elements constituting the molecular design probability vector based on the probability values. The molecular designer 150 may extract (i+1)-th molecular information for designing the (i+1)-th molecule or output a design stop command according to the selected element.

When the molecular designer 150 extracts (i+1)-th molecular information for designing the (i+1)-th molecule, the extracted (i+1)-th molecular information may be again input to the molecular information vectorizer 111, and the processes described above may be repeatedly performed for molecular design until the molecular designer 150 outputs a design stop command.

On the other hand, when the design stop command is output from the molecular designer 150, the i-th molecule may be determined and output as the final molecule.

Hereinafter, an implementation example of designing a final molecule in a deep learning-based molecular design system according to another embodiment of the present invention will be described with reference to FIG. 3B.

In FIG. 3B, the molecular property feature extractor 123 is excluded as compared to FIG. 3A.

The molecular information vectorizer 111 may receive and vectorize i-th molecular information. The surrounding molecular information vectorizer 112 may receive and vectorize surrounding molecular information.

In this case, i-th molecular information vectorized by the molecular information vectorizer 111 and the surrounding molecular information vectorized by the surrounding molecular information vectorizer 112 may be vectorized using the representation method of a molecular graph.

The molecular property information vectorizer 113 may receive and vectorize the molecular property information.

The i-th molecular information vectorized in the molecular information vectorizer 111 may be input to the molecular feature extractor 121. In this case, the molecular information of the i-th molecule may be passed through each of GCNs (Graph Convolutional Networks) six layers including 32, 64, 128, 128, 256, and 256 nodes (or, elements) respectively, and a total of six molecular features of the i-th molecule may be extracted.

The surrounding molecular information from the surrounding molecular information vectorizer 112 may be input to the surrounding molecular feature extractor 122. In this case, the surrounding molecular information may be sequentially passed through a Graph Convolutional Network (GCN) consisting of 128, 128, 128, 128, 128, 128, 128, and 256 nodes (or elements) and a Multi-Layer Perceptron (MLP) consisting of 5 nodes (or elements) to extract the surrounding molecular features.

The molecular property information vectorized by the molecular property information vectorizer 113 and the surrounding molecular feature extracted by the surrounding molecular feature extractor 122 may be input to the integrated feature extractor 130 and concatenated with each other.

The molecular property information and surrounding molecular feature input to the integrated feature extractor 130 and concatenated with each other may be passed through six Multi-Layer Perceptrons (MLPs) including 32, 64, 128, 128, 256, and 256 nodes (or elements), respectively.

The output values that have passed through the six Multi-Layer Perceptrons (MLPs) are added (Sum) with the six features of the i-th molecule extracted by passing through the GCNs (Graph Convolutional Networks), and then input to the next layer of GCNs (Graph Convolutional Networks) or are all concatenated and then passed through one Multi-Layer Perceptron (MLP) consisting of 256 nodes (or elements) to extract the integrated feature of the i-th molecule.

The integrated feature of the i-th molecule extracted by the integrated feature extractor 130 may be input to the molecular design probability calculator 140.

The integrated feature of the i-th molecule input to the molecular design probability calculator 140 may be passed through a Multi-Layer Perceptron (MLP) consisting of 512 nodes (or, elements) and a recurrent neural network (RNN) consisting of 512 nodes (or, elements) to extract a molecular design probability vector for molecular design based on the i-th molecule.

The molecular design probability vector extracted by the molecular design probability calculator 140 may be input to the molecular designer 150.

The molecular designer 150 may calculate probability values by using the elements constituting the input molecular design probability vector as weights respectively and select one of the elements constituting the molecular design probability vector based on the probability values. The molecular designer 150 may extract (i+1)-th molecular information for designing the (i+1)-th molecule or output a design stop command according to the selected element.

When the molecular designer 150 extracts (i+1)-th molecular information for designing the (i+1)-th molecule, the extracted (i+1)-th molecular information may be again input to the molecular information vectorizer 111, and the processes described above may be repeatedly performed for molecular design until the molecular designer 150 outputs a design stop command.

On the other hand, when the design stop command is output from the molecular designer 150, the i-th molecule may be determined and output as the final molecule.

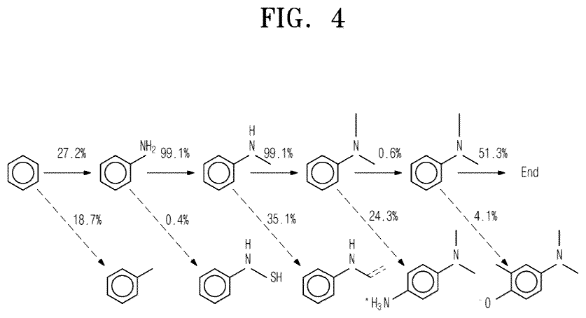

FIG. 4 is a diagram illustrating a process for designing a final molecule according to a molecular design probability vector using benzene as an initial molecule (where i=1) according to an embodiment of the present invention.

Referring to FIG. 4, when the molecular information of an initial molecule, i.e., the initial molecule is input as benzene, the molecular design probability calculator 140 may extract a molecular design probability vector and the molecular designer 150 may design a final molecule using elements constituting the molecular design probability vector.

For example, the molecular design probability calculator 140 may extract a molecular design probability vector for molecular design based on the initial molecule.

The molecular designer 150 may extract molecular information of a second molecule (where i=2) for designing the second molecule according to probability values calculated using the elements constituting the molecular design probability vector.

Referring to FIG. 4, after the probability values for the elements constituting the molecular design probability vector are calculated by the molecular designer 150, examples in which the next molecule is designed based on one of the probability values are indicated by solid arrows, and examples in which the next molecule is not designed are indicated by dotted arrows.

The molecular designer 150 may calculate a probability value using the elements constituting the molecular design probability vector, and design a next molecule according to molecular information corresponding to the largest probability value among probability values.

Alternatively, the molecular designer 150 may calculate the probability values using the elements constituting the molecular design probability vector, and design the next molecule by using the probability values as weights.

Finally, when a design stop command corresponding to 51.3% is output, the molecular designer 150 may stop molecular design and output the final molecule.

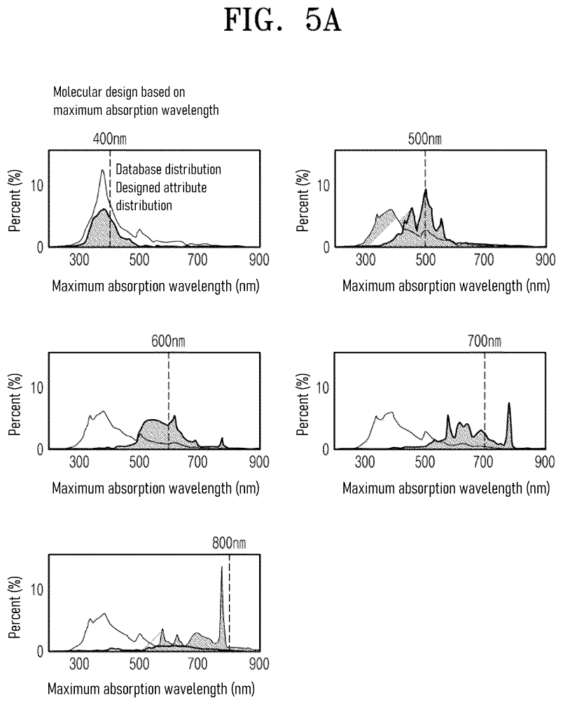

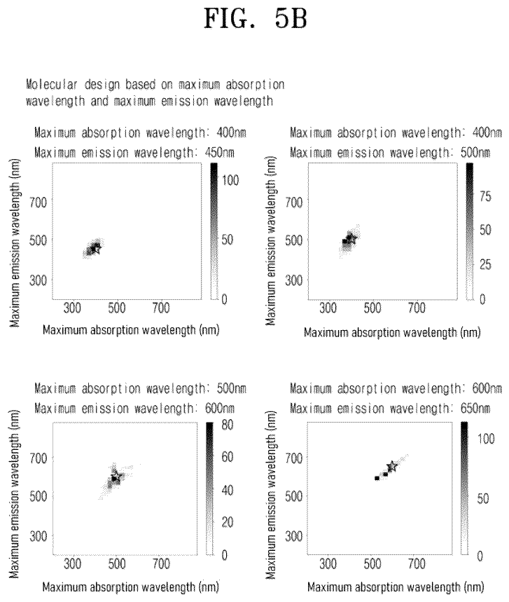

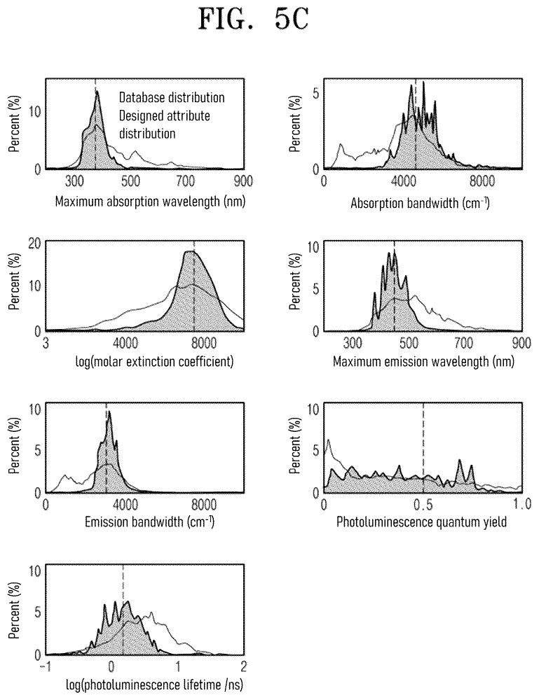

FIG. 5A is a diagram illustrating the result of designing a final molecule based on molecular information, surrounding molecular information, and molecular property information according to an embodiment of the present invention. FIG. 5B is a diagram illustrating the result of designing a final molecule based on molecular information, surrounding molecular information, and molecular property information according to another embodiment of the present invention. FIG. 5C is a diagram illustrating the result of designing a final molecule based on molecular information, surrounding molecular information, and molecular properties information according to still another embodiment of the present invention.

FIG. 5A shows the result of designing the final molecule by performing settings such that the molecular information of the initial molecule does not include chemical structural formula, the surrounding molecular information includes information about toluene, and the molecular property information includes information about the maximum absorption wavelength. In addition, FIG. 5A shows the result of designing the final molecule by repeatedly performing the molecular design described above more than 10000 times.

In the deep learning-based molecular design system 100, when molecular design is performed by setting the maximum absorption wavelength included in the molecular property information to 400 nm, it may be seen that the proportion of final molecules having the maximum absorption wavelength of 400 nm is concentrated around 400 nm compared to a comparison group (database) having the maximum absorption wavelength of 400 nm.

Further, in the deep learning-based molecular design system 100, when molecular design is performed by setting the maximum absorption wavelength included in the molecular property information to 500 nm, it may be seen that the proportion of final molecules having the maximum absorption wavelength of 500 nm is concentrated around 500 nm compared to a comparison group (database) having the maximum absorption wavelength of 500 nm.

Further, in the deep learning-based molecular design system 100, when molecular design is performed by setting the maximum absorption wavelength included in the molecular property information to 600 nm, it may be seen that the proportion of final molecules having the maximum absorption wavelength of 600 nm is concentrated around 600 nm compared to a comparison group (database) having the maximum absorption wavelength of 600 nm.

Further, in the deep learning-based molecular design system 100, when molecular design is performed by setting the maximum absorption wavelength included in the molecular property information to 700 nm, it may be seen that the proportion of final molecules having the maximum absorption wavelength of 700 nm is concentrated around 700 nm compared to a comparison group (database) having the maximum absorption wavelength of 700 nm.

Further, in the deep learning-based molecular design system 100, when molecular design is performed by setting the maximum absorption wavelength included in the molecular property information to 800 nm, it may be seen that the proportion of final molecules having the maximum absorption wavelength of 800 nm is concentrated around 800 nm compared to a comparison group (database) having the maximum absorption wavelength of 800 nm.

In other words, the deep learning-based molecular design system 100 according to an embodiment of the present invention may design a molecule having desired molecular properties with high accuracy by considering the surrounding molecules.

FIG. 5B shows the result of designing a final molecule by performing settings such that the molecular information of the initial molecule has information of a chemical structural formula of benzene, the surrounding molecular information includes information about toluene, and the molecular property information includes information about the maximum absorption wavelength and the maximum emission wavelength. In addition, FIG. 5B shows the result of designing the final molecule by repeatedly performing the molecular design described above more than 10000 times.

When the molecular design is performed in the deep learning-based molecular design system 100 by setting the maximum absorption wavelength included in the molecular property information to 400 nm and the maximum emission wavelength to 450 nm, it may be seen from the proportion of the final molecule that the maximum absorption wavelength is concentrated at 400 nm and the maximum emission wavelength is concentrated at 450 nm.

When the molecular design is performed in the deep learning-based molecular design system 100 by setting the maximum absorption wavelength included in the molecular property information to 400 nm and the maximum emission wavelength to 500 nm, it may be seen from the proportion of the final molecule that the maximum absorption wavelength is concentrated at 400 nm and the maximum emission wavelength is concentrated at 500 nm.

When the molecular design is performed in the deep learning-based molecular design system 100 by setting the maximum absorption wavelength included in the molecular property information to 500 nm and the maximum emission wavelength to 600 nm, it may be seen from the proportion of the final molecule that the maximum absorption wavelength is concentrated at 500 nm and the maximum emission wavelength is concentrated at 600 nm.

When the molecular design is performed in the deep learning-based molecular design system 100 by setting the maximum absorption wavelength included in the molecular property information to 600 nm and the maximum emission wavelength to 650 nm, it may be seen from the proportion of the final molecule that the maximum absorption wavelength is concentrated at 600 nm and the maximum emission wavelength is concentrated at 650 nm.

In other words, the deep learning-based molecular design system 100 according to an embodiment of the present invention may design a molecule having two or more desired molecular properties with high accuracy by considering the surrounding molecules.

Referring to FIG. 5C, a final molecule is designed by configuring the molecular information of an initial molecule to exclude information about the chemical structural formula. In this configuration, the surrounding molecular information includes information about toluene, and the molecular property information includes information about a maximum absorption wavelength (370 nm), absorption bandwidth (4600 cm⁻¹), molar extinction coefficient (4.5), maximum emission wavelength (450 nm), emission bandwidth (3000 cm⁻¹), photoluminescence quantum yield (0.5), and photoluminescence lifetime (1.45 ns).

As shown in FIG. 5C, it may be seen that even when molecular information, surrounding molecular information, and seven pieces of molecular property information are input together at the same time as described above in the deep learning-based molecular design system 100, a final molecule with a dense ratio centered on the input molecular property information is designed.

In other words, the deep learning-based molecular design system 100 according to one embodiment of the present invention may design molecules with various molecular properties with high accuracy by considering the surrounding molecules.

Referring to FIG. 5D, the molecular information of the initial molecule does not include a chemical structural formula, and the molecular property information includes a maximum absorption wavelength (370 nm), an absorption bandwidth (4700 cm⁻¹), a molar extinction coefficient (3.6), a maximum emission wavelength (550 nm), a emission bandwidth (3800 cm⁻¹), a photoluminescence quantum yield (0.01), and photoluminescence lifetime (2.0 ns), and the surrounding molecular information is set to include information about water (H2O) and information about toluene, respectively.

Referring to FIG. 5D, it may be seen that different final molecules are designed when the surrounding molecular information includes information about water and when the surrounding molecular information includes information about toluene.

Specifically, it may be seen that, when the surrounding molecule is water, a molecule with a relatively small Stokes shift is designed due to the large polarity of the solvent, but when the surrounding molecule is toluene, a molecule with a relatively larger donor-acceptor distance is designed due to the small polarity of the solvent.

In other words, it may be seen that the deep learning-based molecular design system 100 according to an embodiment of the present invention may design a molecule having desired molecular properties with high accuracy by considering the surrounding molecules.

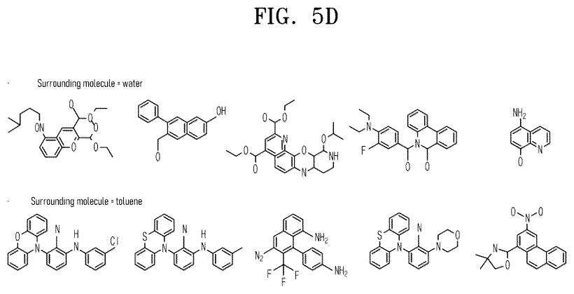

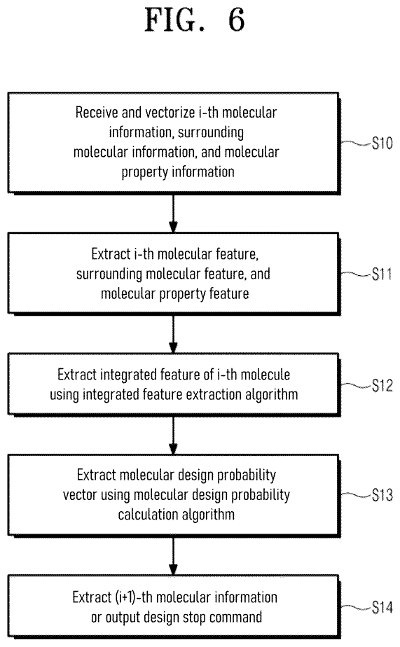

FIG. 6 is a flowchart of a deep learning-based molecular design method according to an embodiment of the present invention.

In step S10, i-th molecular information, surrounding molecular information, and molecular property information may be received and vectorized.

Specifically, the vectorizer 110 may receive and vectorize i-th molecular information, surrounding molecular information, and molecular property information (where i is an integer greater than or equal to 1).

In step S11, a i-th molecular feature may be extracted from the vectorized i-th molecular information, a surrounding molecular feature may be extracted from the vectorized surrounding molecular information, and a molecular property feature may be extracted from the vectorized molecular property information.

Specifically, the molecular feature extractor 121 may extract the i-th molecular feature by inputting the vectorized i-th molecular information into the molecular feature extraction algorithm in the form of a neural network algorithm.

The surrounding molecular feature extractor 122 may extract the surrounding molecular feature by inputting the vectorized surrounding molecular information into the surrounding molecular system feature extraction algorithm in the form of a neural network algorithm.

The molecular property feature extractor 123 may extract the molecular property feature by inputting the vectorized molecular property information into the molecular property feature extraction algorithm in the form of a neural network algorithm.

In step S12, the integrated feature of the i-th molecule may be extracted using an integrated feature extraction algorithm, which is a neural network algorithm that receives the i-th molecular feature, the surrounding molecular feature, and the molecular property feature as inputs.

Specifically, the integrated feature extractor 130 may extract the integrated feature of the i-th molecule by inputting the i-th molecular feature, the surrounding molecular feature, and the molecular property feature, which are provided by the feature extractor 120, into the integrated extractor extraction algorithm in the form of a neural network algorithm.

In step S13, a molecular design probability vector for molecular design may be extracted based on the i-th molecule using the molecular design probability calculation algorithm, which is a neural network algorithm that receives the integrated feature of the i-th molecule as an input.

Specifically, the molecular design probability calculator 140 may extract a molecular design probability vector for molecular design based on the i-th molecule by inputting the integrated feature of the i-th molecule provided by the integrated feature extractor 130 into the molecular design probability calculation algorithm in the form of a neural network algorithm.

In step S14, the (i+1)-th molecular information may be extracted based on the molecular design probability vector, or a design stop command may be output to output the final molecule.

Specifically, the molecular designer 150 may extract (i+1)-th molecular information for designing the (i+1)-th molecule according to probability values calculated using the elements constituting the molecular design probability vector extracted by the molecular design probability calculator 140.

Alternatively, the molecular designer 150 may determine and output the i-th molecule as the final molecule by outputting a design stop command based on the probability values calculated using the elements constituting the molecular design probability vector extracted by the molecular design probability calculator 140.

The foregoing referenced drawings and detailed description of the invention are exemplary of the invention and are intended to illustrate the invention only and are not intended to limit the meaning or the scope of the invention as claimed in the patent. Therefore, it will be understood by those of skill in the art that various changes in form and details may be made without departing from the spirit and scope of the present invention as set forth in the following claims. Accordingly, the technical scope of the present invention should be defined by the accompanying claims.

The embodiments described herein may be implemented with hardware components and software components and/or a combination of the hardware components and the software components. For example, the apparatus, method and components described in the embodiments may be implemented using one or more general-purpose or special purpose computers, such as a processor, a controller and an arithmetic logic unit (ALU), a digital signal processor, a microcomputer, a field programmable array (FPGA), a programmable logic unit (PLU), a microprocessor or any other device capable of executing and responding to instructions.

The processing device may run an operating system (OS) and one or more software applications that run on the OS. The processing device also may access, store, manipulate, process, and create data in response to execution of the software. For convenience of understanding, one processing device is described as being used, but those skilled in the art will appreciate that the processing device includes a plurality of processing elements and/or multiple types of processing elements.

For example, the processing device may include multiple processors or a single processor and a single controller. In addition, different processing configurations are possible, such as parallel processors. The software may include a computer program, a piece of code, an instruction, or some combination thereof, for independently or collectively instructing or configuring the processing device to operate as desired.

Software and/or data may be embodied in any type of machine, component, physical or virtual equipment, computer storage medium or device that is capable of providing instructions or data to or being interpreted by the processing device. The software also may be distributed over network coupled computer systems so that the software is stored and executed in a distributed fashion. In particular, the software and data may be stored by one or more computer readable recording mediums.

The above-described methods may be embodied in the form of program instructions that can be executed by various computer means and recorded on a computer-readable medium. The computer readable medium may include program instructions, data files, data structures, and the like, alone or in combination. Program instructions recorded on the media may be those specially designed and constructed for the purposes of the present invention, or they may be of the kind well-known and available to those having skill in the computer software arts.

Examples of computer readable recording media include magnetic media such as hard disks, floppy disks and magnetic tape, optical media such as CD-ROMs, DVDs, and hardware devices specifically configured to store and execute program instructions, such as ROM, RAM, flash memory, and the like. Examples of program instructions include not only machine code generated by a compiler, but also high-level language code that can be executed by a computer using an interpreter or the like. The hardware device described above may be configured to operate as one or more software modules to perform the operations of the present invention, and vice versa.

Although the embodiments have been described by the limited embodiments and the drawings as described above, various modifications and variations are possible to those skilled in the art from the above description. For example, the described techniques may be performed in a different order than the described method, and/or components of the described systems, structures, devices, circuits, etc. may be combined or combined in a different form than the described method, or other components, or even when replaced or substituted by equivalents, an appropriate result can be achieved. Therefore, other implementations, other embodiments, and equivalents to the claims are within the scope of the following claims.

1. A deep learning-based molecular design system comprising: a vectorizer configured to receive and vectorize i-th molecular information, surrounding molecular information, and molecular property information;
a feature extractor configured to extract a molecular feature from the vectorized the i-th molecular information, extract a surrounding molecular feature from the vectorized surrounding molecular information, and extract a molecular property feature from the vectorized molecular property information;
an integrated feature extractor configured to extract an integrated feature of the i-th molecule using an integrated feature extraction algorithm which is a neural network algorithm that receives the i-th molecular property feature, the surrounding molecular feature, and the molecular property feature as an input;
a molecular design probability calculator configured to extract a molecular design probability vector for molecular design based on the i-th molecule using a molecular design probability calculation algorithm, which is a neural network algorithm that receives the integrated feature of the i-th molecule as an input; and
a molecular designer configured to extract (i+1)-th molecular information based on the molecular design probability vector or output a design stop command to output a final molecule,
wherein i is an integer greater than or equal to 1.

2. The deep learning-based molecular design system of claim 1, wherein the vectorizer includes:
a molecular information vectorizer configured to receive the i-th molecular information in form of SMILES (Simplified molecular-Input Line-Entry System) representation, and vectorize the molecular information using at least one of a molecular fingerprint, a molecular descriptor, an image of a chemical structural formula, a molecular graph, molecular coordinates, and a SMILES code;
a surrounding molecular information vectorizer configured to receive the i-th surrounding molecular information in the form of the SMILES (Simplified molecular-Input Line-Entry System) representation, and vectorize the surrounding molecular information using at least one of the molecular fingerprint, the molecular descriptor, the image of a chemical structural formula, the molecular graph, the molecular coordinates, and the SMILES code; and
a molecular property information vectorizer configured to receive the i-th molecular property information in form of a string or a set of real values and vectorize the molecular property information using at least one of tokenization, normalization, and one-hot encoding.

3. The deep learning-based molecular design system of claim 2, wherein the feature extractor includes:
a molecular feature extractor configured to extract the i-th molecular feature using a molecular feature extraction algorithm, which is a neural network algorithm that receives the vectorized i-th molecular information as an input;
a surrounding molecular feature extractor configured to extract a surrounding molecular feature using a surrounding molecular feature extraction algorithm, which is a neural network algorithm that receives the vectorized surrounding molecular information as an input; and
a molecular property feature extractor configured to extract the molecular property feature using a molecular property feature extraction algorithm, which is a neural network algorithm that receives the vectorized molecular property information as an input.

4. The deep learning-based molecular design system of claim 1, wherein the molecular information includes information about a chemical structural formula,
wherein the surrounding molecular information includes information about one or more solvents, and
wherein the molecular property information includes information about at least one of structural, chemical, physical, spectroscopic, electrochemical, and reactivity of the molecule.

5. The deep learning-based molecular design system of claim 4, wherein molecular information of an initial molecule (where i=1) includes information about a chemical structural formula provided by a user or provided by the deep learning-based molecular design system.

6. The deep learning-based molecular design system of claim 1, wherein the molecular designer is configured to extract (i+1)-th molecular information for designing the (i+1)-th molecule according to a probability value calculated using one of elements constituting the molecular design probability vector; and
wherein (i+1)-th molecular information includes information about the chemical structural formula of the (i+1)-th molecule, which is designed by either binding a single atom to one of atoms constituting the i-th molecule, or by adding a bond that connects the atoms constituting the i-th molecule.

7. The deep learning-based molecular design system of claim 1, wherein the molecular designer is configured to output the design stop command according to a probability value calculated using one of the elements constituting the molecular design probability vector to determine the i-th molecule as the final molecule.

8. The deep learning-based molecular design system of claim 3, wherein the molecular feature extraction algorithm, the surrounding molecular feature extraction algorithm, the molecular property feature extraction algorithm, the integrated feature extraction algorithm, and the molecular design probability calculation algorithm include at least one hidden layer.

9. A deep learning-based molecular design method comprising:
receiving and vectorizing, by a vectorizer, i-th molecular information, surrounding molecular information, and molecular property information;
extracting, by a feature extractor, an i-th molecular feature from the vectorized the i-th molecular information, extracting a surrounding molecular feature from the vectorized surrounding molecular information, and extracting a molecular property feature from the vectorized molecular property information;
extracting, by an integrated feature extractor, an integrated feature of the i-th molecule using an integrated feature extraction algorithm which is a neural network algorithm that receives the i-th molecular feature, the surrounding molecular feature, and the molecular property feature as an input;
extracting, by a molecular design probability calculator, a molecular design probability vector for molecular design based on the i-th molecule using a molecular design probability calculation algorithm, which is a neural network algorithm that receives the integrated feature of the i-th molecule as an input; and
extracting, by a molecular designer, (i+1)-th molecular information based on the molecular design probability vector or outputting a design stop command to output a final molecule,
wherein i is an integer greater than or equal to 1,

10. A non-transitory computer-readable recording medium having recorded thereon a program for executing the deep learning-based molecular design method of claim 9.
